(19) United States
(12) Patent Application Publication
(10) Pub. No.: US 2024/0216098 A1
(43) Pub. Date: Jul. 4, 2024

(54) COVER FOR MEDICAL DEVICE, SYSTEM AND METHOD THEREOF

(71) Applicant: Coherex Medical, Inc., Salt Lake City, UT (US)

(72) Inventors: Sung K. Lee, Salt Lake City, UT (US); Tom Ditter, Mission Viejo, CA (US)

(21) Appl. No.: 18/090,077

(22) Filed: Dec. 28, 2022

(51) Int. Cl. A61B 46/23; A61B 46/00

(52) U.S. Cl. CPC A61B 46/23; A61B 46/40

(57) ABSTRACT

A medical device system configured to occlude an opening in a heart, the medical device system including a delivery system and an implant. The delivery system including a handle and a catheter, the implant removably coupled to the catheter. The implant includes a frame work and a tissue growth member. The tissue growth member includes an inner sheet, an outer sheet, and a reinforcement textile. The inner sheet is positioned along an inner side of the framework and the outer sheet is positioned along an outer side of the framework. The reinforcement textile is positioned between the inner and outer sheets to define a texture along an outer surface of the tissue growth member.

### TECHNICAL FIELD

The present invention relates generally to the occlusion of tissue openings or appendages and, more specifically, to devices, systems, and methods for reinforcing a cover for an occlusion device.

### BACKGROUND

The upper chambers of the heart, the atria, have appendages attached to each of them. For example, the left atrial appendage is a feature of all human hearts. The physiologic function of such appendages is not completely understood, but they do act as a filling reservoir during the normal pumping of the heart. The appendages typically protrude from the atria and cover an external portion of the atria. Atrial appendages differ substantially from one to another. For example, one atrial appendage may be configured as a tapered protrusion while another atrial appendage may be configured as a re-entrant, sock-like hole. The inner surface of an appendage is conventionally trabeculated with cords of muscular cardiac tissue traversing its surface with one or multiple lobes.

The atrial appendages appear to be inert while blood is being pumped through them during normal heart function. In other words, the appendages don't appear to have a noticeable effect on blood pumped through them during normal heart function. However, in cases of atrial fibrillation, when the atria go into arrhythmia, blood may pool and thrombose inside of the appendages. Among other things, this can pose a stroke risk when it occurs in the left appendage since the thrombus may be pumped out of the heart and into the cranial circulation once normal sinus rhythm is restored following arrhythmia events.

Historically, appendages have sometimes been modified surgically to reduce the risk imposed by atrial fibrillation. In recent years devices which may be delivered percutaneously into the left atrial appendage have been introduced. The basic function of these devices is to exclude the volume within the appendage with an implant which then allows blood within the appendage to safely thrombose and then to be gradually incorporated into cardiac tissue. This process, coupled with the growth of endothelium over the face of the device, can leave a smooth, endothelialized surface where the appendage is located. In comparison to surgical procedures, devices implanted percutaneously are a less invasive means for addressing the problems associated with the left atrial appendage.

During implantation of the device the physician typically uses a sound transmitting instrument, such as, transesophageal echocardiography (TEE) to monitor the location of the device during the procedure. However, the materials often used to form the cover of the device for blocking openings in the anatomy, such as the left atrial appendage, are formed as polymeric layers and are attached to a frame of the device. The polymeric layers or materials often used for device covers are expanded polytetrafluoroethylene (ePTFE) and/or polyurethane foam, which exhibit properties that promote endothelialization and tissue growth over and within the cover, but also exhibit properties of hydrophobicity and porosity that may trap air within and between the layers of foam and ePTFE. The trapped air can prevent sound energy of the TEE instrument to transmit through the device and emit a complete structural image of the device to the physician, thus making it difficult for the physician to determine and monitor the location of the device during implantation. This misdiagnosis of location may result in positioning the device in a less than optimal position and/or orientation than what is intended and may increase the risk of effusion. Further, the manufacturing steps for forming and attaching the polymeric cover to the frame of the device are complex, result in handling issues of the device, and can be laborious. As such, it would be advantageous for there to be an alternative device cover of the type that blocks openings within the anatomy and that provides similar advantages of ePTFE and polyurethane foam of promoting endothelialization, but also substantially eliminates the challenges of using these materials.

### BRIEF SUMMARY OF THE INVENTION

The present invention is directed to a medical device system configured to occlude an opening in a heart. In one embodiment, the medical device system includes a delivery system and an implant. The delivery system includes a handle and a catheter, and the implant is removably coupled to the catheter. The implant includes a framework and a tissue growth member. The tissue growth member includes an inner sheet, an outer sheet, and a reinforcement textile. The inner sheet is positioned along an inner side of the framework, and the outer sheet is positioned along an outer side of the framework. The reinforcement textile is positioned between the inner and outer sheets to define a texture along an outer surface of the tissue growth member.

In another embodiment, the reinforcement textile extends with longitudinal structures and lateral structures, each one of the lateral structures extending transverse relative to each one of the longitudinal structures. In another embodiment, the reinforcement textile extends with longitudinal structures and lateral structures, each one of the longitudinal structures being spaced relative to adjacently extending ones of the longitudinal structures, each one of the lateral structures being spaced relative to adjacently extending ones of the lateral structures. In another embodiment, the reinforcement textile extends with longitudinal structures and lateral structures, the longitudinal structures positioned over the framework to extend radially outward from a proximal hub of the framework toward a distal end of the framework, the lateral structures positioned over the framework to extend transverse relative to the longitudinal structures. In still another embodiment, the reinforcement textile extends with longitudinal structures and lateral structures, each one of the longitudinal structures extending over and under different ones of the lateral structures, each one of the lateral structures extending over and under different ones of the longitudinal structures.

In another embodiment, the outer surface of the tissue growth member defines recesses therein such that structure that defines the recesses also defines the texture, the recesses being sized and configured to promote endothelialization along the outer surface of the tissue growth member. In yet another embodiment, the reinforcement textile and the outer sheet extend to define a laminated structure, the laminated structure extending with the texture along the outer surface of the tissue growth member. In another embodiment, the tissue growth member includes an intermediate sheet, the intermediate sheet positioned along the outer side of the framework and below the reinforcement textile.

In accordance with another embodiment of the present invention, a medical device configured to be percutaneously delivered with a delivery system to occlude an opening in a heart is provided. The medical device includes a framework and a tissue growth member. The framework extends radially outward from a proximal hub to a distal side of the framework. The tissue growth member is attached to the framework, the tissue growth member including an outer polymeric sheet, and a reinforcement textile. The reinforcement textile includes longitudinal structures and lateral structures, the longitudinal structures radially spaced from each other and each extending over the framework from the proximal hub toward the distal side of the framework. The lateral structures are spaced from each other and positioned laterally relative to the longitudinal structures to each extend radially around a circumference of the radially extending framework. The outer polymeric sheet is positioned over the reinforcement textile so that the longitudinal structures and the lateral structures define a texture along an outer surface of the tissue growth member.

In another embodiment, each one of the longitudinal structures extend over and under different ones of the lateral structures, and wherein each one of the lateral structures extend over and under different ones of the longitudinal structures. In still another embodiment, the outer surface of the tissue growth member defines recesses therein such that structure that defines the recesses also defines the texture, the recesses being sized and configured to promote endothelialization along the outer surface of the tissue growth member. In another embodiment, the reinforcement textile and the outer polymeric sheet extend to define a laminated structure, the laminated structure extending with the texture along the outer surface of the tissue growth member.

In another embodiment, the tissue growth member includes an inner polymeric sheet, the inner polymeric sheet positioned along an inner side of the framework. In another embodiment, the tissue growth member includes an inner polymeric sheet and an intermediate polymeric sheet, the intermediate polymeric sheet and the outer polymeric sheet positioned along opposite sides of the reinforcement textile.

In accordance with another embodiment of the present invention, a method for occluding an opening in a heart is provided. The method includes the steps of: advancing a medical device through a vasculature toward the opening in the heart with a pusher catheter such that the medical device is coupled adjacent a distal end of the pusher catheter; positioning the medical device in the opening in the heart such that the medical device includes a framework and a tissue growth member, the tissue growth member including an outer polymeric sheet and a reinforcement textile, the reinforcement textile including longitudinal structures and lateral structures, the longitudinal structures radially spaced from each other and each extending over the framework from the proximal hub toward the distal side of the framework, the lateral structures spaced from each other and positioned laterally relative to the longitudinal structures to each extend radially around a circumference of the radially extending framework, the outer polymeric sheet positioned over the reinforcement textile so that the longitudinal structures and the lateral structures define a texture along an outer surface of the tissue growth member; and anchoring the medical device within the opening of the heart with tines extending from the framework so that the texture of the tissue growth member is positioned against tissue defining the opening in the heart.

In another embodiment, the positioning step includes positioning the medical device such that the tissue growth member includes each one of the longitudinal structures to extend over and under different ones of the lateral structures, and each one of the lateral structures to extend over and under different ones of the longitudinal structures. In another embodiment, the anchoring step includes positioning recesses defined in the outer surface of the tissue growth member against tissue in the heart. In another embodiment, the positioning step includes positioning the medical device with a laminated structure extending with the texture along the outer surface of the tissue growth member, the laminated structure extending with at least the reinforcement textile and the outer polymeric sheet.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1 is a perspective view of a medical device system, depicting an implant coupled to a delivery system, according to one embodiment of the present invention;

FIG. 1A is a partial cross-sectional view of the medical device system taken along section line 1A-1A of FIG. 1, depicting the implant in a deployed position, according to another embodiment of the present invention;

FIG. 2 is a perspective view of the medical device system, depicting the implant in a partially deployed position, according to another embodiment of the present invention;

FIG. 2A is a partial cross-sectional view of the medical device system taken along section line 2A-2A of FIG. 2, depicting anchors of the implant in a retracted position, according to another embodiment of the present invention;

FIG. 3 is a perspective view of the medical device system, the implant being at least partially constricted within a sheath of the medical device system, according to another embodiment of the present invention;

FIG. 4 is a side view of components of the implant, depicting a first polymer sheet positioned on a mandril and a framework of the implant positioned above the first sheet, according to another embodiment of the present invention;

FIG. 5 is a side view of components of the implant, depicting the framework and first polymer sheet positioned on the mandril and a reinforcement textile and a second polymer sheet positioned above the framework, according to another embodiment of the present invention;

FIG. 6 is a side view of an occluder portion of the implant, depicting the occluder portion with a textured surface subsequent to a lamination process, according to another embodiment of the present invention;

FIG. 7 is a perspective view of the implant, depicting an anchor portion assembled with the occluder portion with the textured surface, according to another embodiment of the present invention;

FIG. 8 is a simplified enlarged cross-sectional view of the tissue growth member taken from region A of FIG. 1A, depicting a reinforcement textile extending between the framework and a polymer sheet, according to another embodiment of the present invention;

FIG. 9 is a side view of another embodiment of an occluder portion of an implant, depicting a framework and a first polymer sheet positioned on a mandril and a second polymer sheet, a reinforcement textile, and a third polymer sheet positioned above the framework, according to the present invention; and

FIG. 10 is a simplified enlarged cross-sectional view of another embodiment of the tissue growth member, depicting the reinforcement textile extending between the second polymer sheet and the third polymer sheet, according to the present invention.

### DETAILED DESCRIPTION OF THE INVENTION

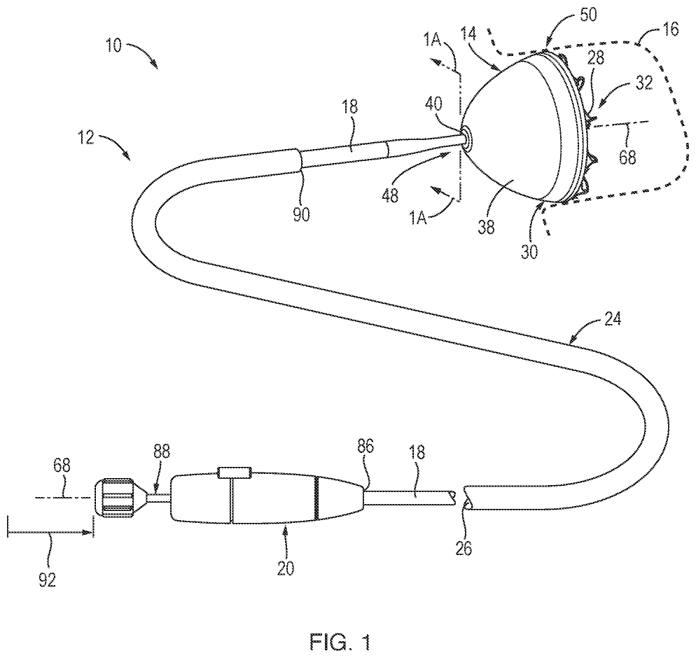

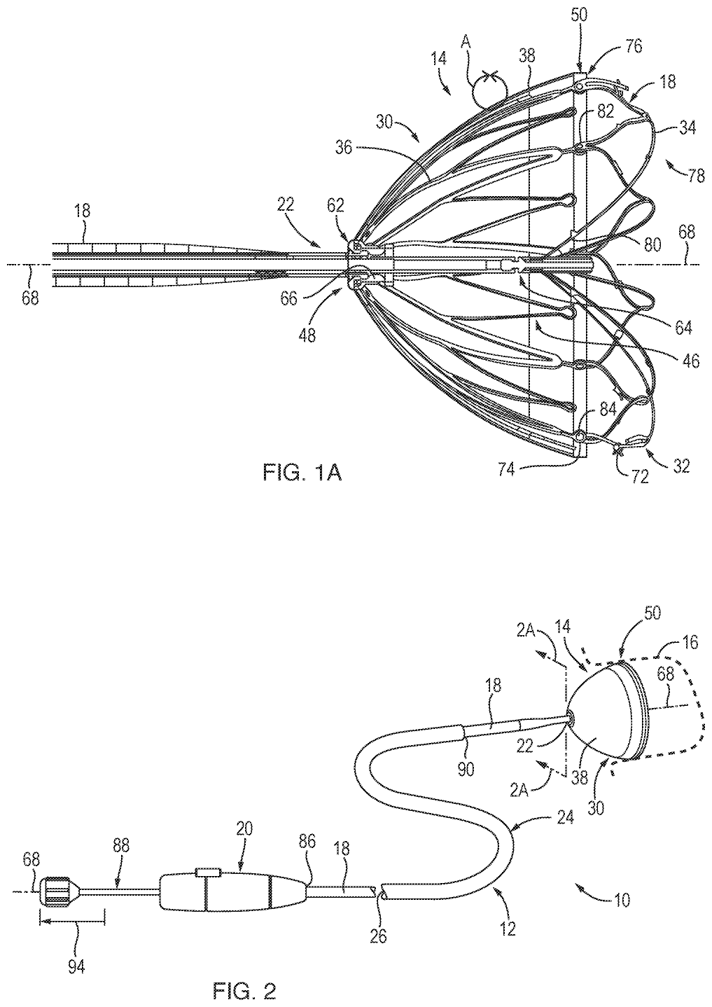

Referring to FIGS. 1 and 1A, a medical device system 10 including a delivery system 12 and an implant 14 is provided. The medical device system 10 may be employed in interventional procedures for percutaneously closing and modifying an opening such as, for example, a left atrial appendage 16 within a heart (not shown). The delivery system 12 may include a pusher catheter 18 and a handle 20, the pusher catheter 18 being coupled to the implant 14 adjacent a distal end 22 of the pusher catheter 18. Also, the delivery system 12 may include a sheath 24 sized and configured to facilitate advancing the implant 14 through a lumen 26 of the sheath 24 with the pusher catheter 18 (see also FIG. 3). In addition, the implant 14 may extend to define a framework 28 extending along an occluder portion 30 and an anchor portion 32. The anchor portion 32 may extend with an anchor frame 34 and the occluder portion 30 may include an occluder frame 36 with a tissue growth member 38 attached to the occluder frame 36. With reference to FIGS. 1 and 5-8, the tissue growth member 38 may be sized and configured with enhanced structural strength with, for example, one or more polymer sheets and a reinforcement textile 44. Such reinforcement textile 44 may be positioned under at least one of the polymer sheets and laminated therewith to define a texture 58 along an outer surface 110 of the tissue growth member 38. With this arrangement, the texture 58 defined along the outer surface 110 may define recesses 60 therein, the recesses 60 being sized and configured to promote endothelialization over the outer surface of the tissue growth member 38.

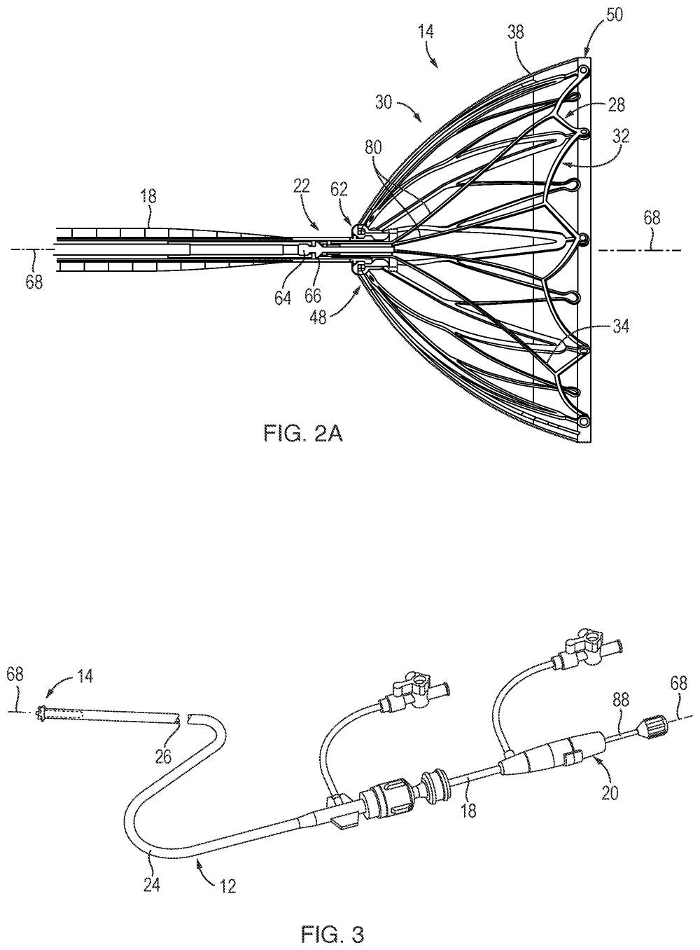

Now with reference to FIGS. 1, 1A, 2, and 2A, as previously set forth, the medical device system 10 including the delivery system 12 and implant 14 may be configured such that the implant 14 may be removably coupled to the delivery system 12. The medical device system 10 may be employed in interventional procedures for percutaneously closing and modifying an opening such as, for example, the left atrial appendage 16 within a heart (not shown). The delivery system 12 may include the pusher catheter 18 and the handle 20, the pusher catheter 18 being coupled to the implant 14 adjacent the distal end 22 of the pusher catheter 18. Also, the delivery system 12 may include the sheath 24 sized and configured to facilitate advancing the implant 14 through the lumen 26 of the sheath 24 with the pusher catheter 18 (see also FIG. 3). In addition, the implant 14 may extend to define the framework 28 extending along the occluder portion 30 and anchor portion 32. The anchor portion 32 may extend with the anchor frame 34 and the occluder portion 30 may include the occluder frame 36 where the tissue growth member 38 may be attached to the occluder frame 36. The implant 14 may include a primary hub 62 and a secondary hub 64 such that the framework 28 may extend between the primary hub 62 and the secondary hub 64. The primary hub 62 may define a bore hub 66 and an axis 68 that each may extend through the primary hub 62 such that the axis 68 may extend axially relative to the bore hub 66 and structure of the primary hub 62. Such axis 68 may also extend axially along a length of the delivery system 12 and the components thereof. Further, secondary hub 64 may be moveable along the axis 68 through the primary hub 62 so as to move the framework 28 between a constricted position (FIG. 2A) and a deployed position (FIG. 1A). With this arrangement, as the secondary hub 64 moves along the axis 68 and through the bore hub 66 of the primary hub 62 such that portions of the framework 28 may be minimized as the framework 28 moves between the constricted and deployed positions. Furthermore, upon the framework 28 at least partially being constricted within the sheath 24, the framework 28 may extend with structure that may minimize resistance with an internal surface of the sheath 24.

As previously set forth, the framework 28 may extend with the occluder portion 30 to define the occluder frame 36 and the framework 28 may extend with the anchor portion 32 to define the anchor frame 34. The anchor frame 34 may extend with the anchor portion 32 and define anchor tines 72 extending therefrom. The occluder frame 36 may extend with the occluder portion 30 with the tissue growth member 38 attached to the occluder frame 36. The tissue growth member 38 may be in the form of an occlusive member, but may also be in the form of a filter member, a mesh member, a membrane or any other structure, or combinations thereof, sized and configured to promote tissue in-growth thereto. Further, in one embodiment, the tissue growth member 38 may be formed from one or more polymeric materials. In another embodiment, the tissue growth member may be formed from one or more polymeric materials and/or a fabric. For example, the first and second polymer sheets 40, 42 (FIG. 5) may be formed from the same polymeric material or different polymeric materials and the reinforcement textile 44 (FIG. 5) may be formed from a fabric. Even further, the tissue growth member 38 may be sized and configured to extend with multiple layers, such as, first and second polymer sheets 40, 42 (FIG. 5) and the reinforcement textile 44 (FIG. 5) may be configured to be relatively thin such that the tissue growth member 38 being formed on the implant 14 may be readily constricted within the sheath 24.

Further, the occluder frame 36 may be coupled to the primary hub 62 such that the occluder frame 36 may extend radially outward from the primary hub 62 and may extend distally to an occluder distal end 50 of the occluder frame 36. Adjacent to the occluder distal end 50, the occluder frame 36 may include multiple occluder frame eyelets 74 defined in the occluder frame 36. The anchor frame 34 may extend between a first anchor frame end 76 and a second anchor frame end 78, the first anchor frame end 76 coupled to the occluder frame 36 and the second anchor frame end 78 coupled to the secondary hub 64. The anchor frame 34 may extend with multiple anchor frame segments 80, interconnected to each other, extending between the first and second anchor frame ends 76, 78, of the anchor frame 34. Adjacent to the first anchor frame end 76 of the anchor frame 34, the anchor frame 34 may include multiple anchor frame eyelets 82 along multiple ones of the anchor frame segments 80 of the anchor frame 34. At the secondary hub 64, multiple ones of the anchor frame segments 80 or anchor frame extensions may be coupled to the secondary hub 64. Each of the occluder frame eyelets 74 may be coupled to a corresponding one of the anchor frame eyelets 82 with a hinge component 84. The hinge component 84 may extend through the occluder frame eyelets 74 and the anchor frame eyelets 82 so as to facilitate the anchor frame 34 to pivot about the hinge component 84 so as to pivot or rotate relative to the occluder frame 36. With this arrangement, the anchor frame 34 may be pivotably coupled (or hingeably coupled) to the occluder frame 36 so that the anchor frame 34 may move between a retracted position (FIG. 2A) and a deployed position (FIG. 1A). The retracted position of the anchor frame 34 may also be an anchor constrained position or pivoted position. As such, the pivoting between the retracted and deployed positions of the anchor frame 34 may assist a physician in adjusting the position of the implant 14 subsequent to the anchor portion 32 being secured to tissue in, for example, the left atrial appendage 16.

With reference to FIGS. 1-3, as previously set forth, the implant 14 may be delivered through the vasculature with the delivery system 12. The delivery system 12 may include the pusher catheter 18 and the handle 20, the handle 20 integrated with a proximal portion 86 of the pusher catheter 18. The handle 20 may include various functional components, such as an anchor actuator 88, to manipulate the anchor frame 34 between the deployed position (FIG. 1A) and the retracted position (FIG. 2A). The delivery system 12 may include and be employed with the sheath 24 for delivering the implant 14 through the vasculature and to the left atrial appendage 16 in the heart. The sheath 24 may be positioned within the vasculature using known interventional techniques with a sheath distal end 90 to be positioned adjacent the left atrial appendage 16 of the heart. Upon the implant 14 being advanced through the lumen 26 of the sheath 24 to the sheath distal end 90 (the implant 14 being in the constricted position partially shown in dashed lines adjacent the sheath distal end 90 (see FIG. 3)), the implant 14 may at least partially be deployed from the sheath 24. That is, the sheath 24 may then be manually moved proximally (and/or the pusher catheter 18 advanced distally) so that the occluder portion 30 of the implant 14 may be deployed from the sheath distal end 90. Such occluder portion 30 may immediately self-expand as the occluder portion 30 is exposed from the sheath distal end 90. At this stage, the implant 14 may be in a partially deployed state, after which, the implant 14 may be moved to a fully deployed state by deploying the anchor portion 32 (see FIG. 1). For example, upon the occluder portion 30 initially being deployed, the anchor portion 32 may be in the retracted position with the anchor actuator 88 of the handle 20 in the proximal position. Once a physician determines that the occluder portion 30 is in an appropriate and desired position adjacent the left atrial appendage 16, the anchor portion 32 may be pivoted from the retracted position to the deployed position by moving the anchor actuator 88 to the distal position, as shown by arrow 92 (see FIG. 1). Once the anchor portion 32 is moved to the deployed position, the anchor tines 72 (FIG. 1A) of the anchor portion 32 may engage tissue to secure the implant 14 in the left atrial appendage 16. If the physician determines that the implant 14 is not in an optimal secured position in the left atrial appendage 16, the anchor portion 32 may be pivoted back to the retracted position by moving the anchor actuator 88 from the distal position to the proximal position, as shown by arrow 94 in FIG. 2. As such, the anchor actuator 88 may be manually moved proximally and distally to move the anchor portion 32 between the retracted and deployed positions such that the anchor portion 32 pivots between the deployed and retracted positions. In this manner, the anchor portion 32 of the implant 14 may be secured and disengaged from tissue in the left atrial appendage 16 as needed by the physician until the physician obtains an optimal position or is satisfied with its position prior to releasing the delivery system 12 from the implant 14. Additional disclosure of a similar medical device delivery system is disclosed in commonly assigned U.S. patent application Ser. No. 15/438,650, filed on Feb. 21, 2017, now issued as U.S. Pat. No. 10,631,969, entitled MEDICAL DEVICE FOR MODIFICATION OF LEFT ATRIAL APPENDAGE AND RELATED SYSTEMS AND METHODS, the disclosure of which is incorporated by reference herein in its entirety.

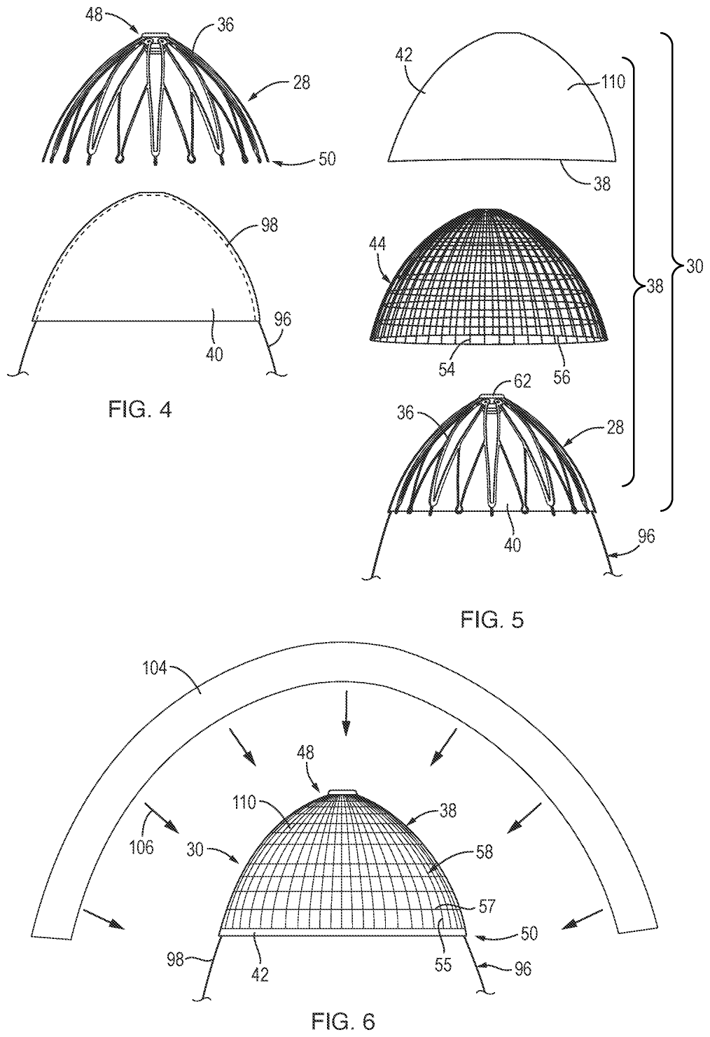

With reference now to FIGS. 4-7, in one embodiment, a method for forming the tissue growth member 38 with the framework 28 so as to form the occluder portion 30 will now be described. As previously set forth, the tissue growth member 38 may include the first polymer sheet 40 and the second polymer sheet 42 with the reinforcement textile 44 therebetween. The reinforcement textile 44 may also be referenced as a reinforcement structure. In some embodiments, each layer or portion, such as the first and second polymer sheets 40, 42, may include multiple layers with varying thicknesses. Further, the first and second polymer sheets 40, 42 may be formed from a polyurethane material, a polyethylene terephthalate (“PET”) material, a silicon material, or a copolymer thereof, or any other suitable biocompatible polymeric material. Such tissue growth member 38 may be coupled to the framework 28 by employing a lamination process. For example, with respect to FIG. 4, the first polymer sheet 40 may be appropriately sized and positioned over a mandril 96. The mandril 96 may define a tip portion 98 (shown with dashed lines) with a dome shape sized to correspond generally to the size and shape of an inner side 46 (FIG. 8) of the implant 14.

As depicted in FIGS. 4 and 5, the occluder frame 36 of the framework 28 of the occluder portion 30 may be positioned above and over the tip portion 98 of the mandril 96 to rest over the first polymer sheet 40 such that the inner side of the occluder frame 36 may extend with the first polymer sheet 40. The framework 28 may be a super-elastic material, such as Nitinol, so that the framework 28 may self-expand from a constricted state to a non-constricted state. As such, upon positioning the occluder frame 36 over the mandril 96 and over the first polymer sheet 40, the occluder frame 36 may be in a non-constricted state or formed state such that the framework 28 may previously undergo a heat-setting process to form the super-elastic material in such non-constricted state or the position that the framework will automatically self-expand to, such as the general position of the occluder frame 36 shown in FIGS. 4 and 5. Further, the occluder frame 36 may be positioned over the mandril 96 so that the first polymer sheet 40 extends from the proximal side 48 of the implant 14 toward and adjacent to the occluder distal end 50 of the occluder frame 36. As depicted, the framework 28 positioned over the mandril 96 may only include the occluder frame 36 positioned over the first polymer sheet 40. The anchor frame 34 (FIG. 1A) may be assembled to the occluder frame 36 at a later step, as set forth below. In another embodiment, the occluder frame 36 may be coupled to the anchor frame 34 prior to positioning the occluder frame 36 over the first polymer sheet 40. In another embodiment, the anchor frame 34 may extend, at least partially, over a portion of the first polymer sheet 40.

Now with reference to FIGS. 5 and 6, as previously set forth, the tissue growth member 38 may also include the second polymer sheet 42 and the reinforcement textile 44. The second polymer sheet 42 may be positioned over an outer side 52 of the occluder frame 36 such that the second polymer sheet 42 may extend from the proximal side 48 of the occluder frame 36 to adjacent the occluder distal end 50, as depicted in FIG. 6, with the reinforcement textile 44 thereunder. As such, the reinforcement textile 44 may be positioned between the outer side of the occluder frame 36 of the implant 14 and the second polymer sheet 42.

The reinforcement textile 44 may be sized and configured to reinforce and strengthen the tissue growth member 38 itself as well as reinforce and strengthen the coupling of the tissue growth member 38 to the framework 28 of the occluder portion 30. Further, the reinforcement textile 44 may be sized and configured to be positioned over the occluder frame 36, and directly against the occluder frame 36. Upon positioning the reinforcement textile 44 over an outer side 52 of the occluder frame 36, the reinforcement textile 44 may be oriented and positioned to define longitudinal structures 54 and lateral structures 56. The longitudinal structures 54 may be elongated structures that extend radially outward from the primary hub 62 or the proximal side 48 of the occluder frame 36 to adjacent the occluder distal end 50 of the occluder frame 36. The lateral structures 56 may also be elongated structures that may be oriented to extend laterally relative to the longitudinal structures 54 so as to extend adjacently around a circumference 104 of the occluder frame 36. The longitudinal structures 54 may include multiple ones of the elongated structures that may be spaced relative to adjacently extending longitudinal structures 54. Likewise, the lateral structures 56 may include multiple ones of the elongated structures that may be spaced relative to adjacently extending lateral structures 56. As such, the combination of the longitudinal structures 54 and the lateral structures 56 may extend in a matrix or grid like pattern or an array of rows and columns radially extending along the outer side 52 of the occluder frame 36. Further, each one of the longitudinal structures 54 may extend over and under every other one of the lateral structures 56 so as to be weaved with the lateral structures 56 (see FIG. 8). Similarly, each one of the lateral structures 56 may extend over and under every other one of the longitudinal structures 54 so as to be weaved with the longitudinal structures 54 (see FIG. 8). In another embodiment, the longitudinal and lateral structures 54, 56 may extend with a radial curve along the outer side 52 of the occluder frame 36. In another embodiment, the longitudinal and/or the lateral structures 54, 56 may extend along the radial curve of the outer side 52 of the occluder frame 36 and may extend with a wavy profile along their respective length. As previously set forth, the reinforcement textile 44 may be formed from a fabric material, the fabric material being formed from a natural fiber or a polymeric fiber, such as PET, or combinations thereof. In another embodiment, the reinforcement textile 44 may be formed of a polymeric material, such as PET or any suitable biocompatible polymeric material. In still another embodiment, the reinforcement textile 44 may be formed from polymeric materials, such as polyester based materials, polytetrafluoroethylene, polyurethane, nylon, PET and/or related copolymers thereof. In another embodiment, the reinforcement textile 44 may be formed with an electrospinning process using any one or combinations of the polymeric materials set forth herein, as known to one skilled in the art, so as to exhibit a random structure. In another embodiment, the reinforcement textile 44 may be formed from metal wires, such as Nitinol. In another embodiment, the reinforcement textile 44 may be integrated with the framework 28 itself to form patterns therewith. In another embodiment, the reinforcement textile 44 may be formed with a fabric or ePTFE, or a combination thereof, or any other polymeric material that may be employed with or as a fiber. In another embodiment, the reinforcement textile 44 may be formed with metal films.

As previously set forth, the reinforcement textile 44 may be sized and configured to be positioned directly over the occluder frame 36 so as to be oriented to be positioned against the occluder frame 36 in an aligned manner. In one embodiment, the reinforcement textile 44 may be glued directly to the occluder frame 36. Once the reinforcement textile 44 is positioned, the second polymer sheet 42 may be positioned directly over and against an outer side of the reinforcement textile 44 so as to appropriately oriented and aligned with the first polymer sheet 40, the occluder frame 36 and the reinforcement textile 44, as depicted in FIG. 6.

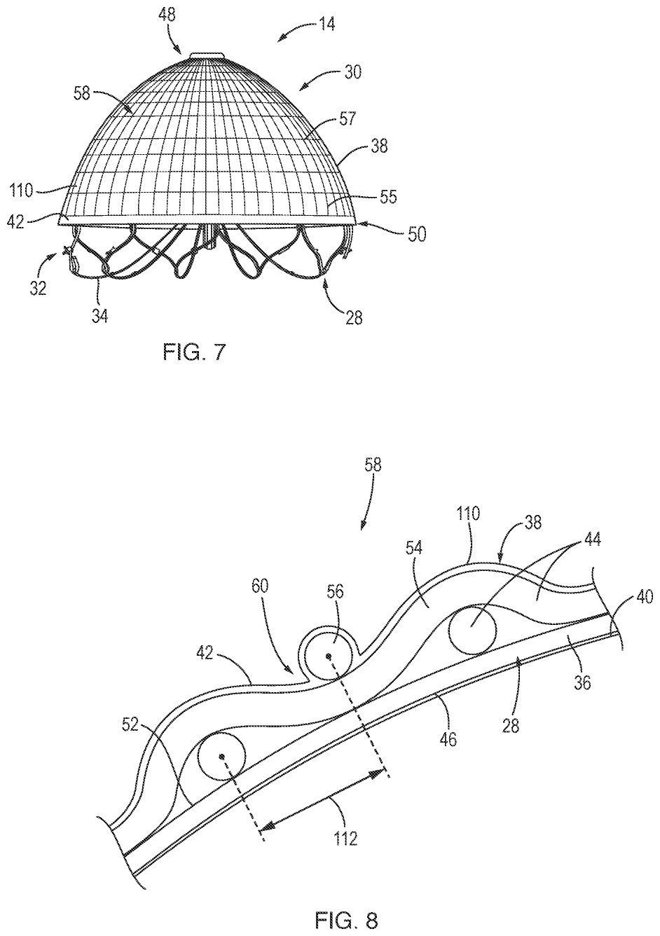

Now with reference to FIG. 6, with the occluder frame 36 and tissue growth member 38 positioned on the tip portion 98 of the mandril 96, the tissue growth member 38 may be heated to laminate the layers of the tissue growth member 38 together and to the occluder frame 36. For example, the tissue growth member 38 may be heated by the mandril 96 conducting heat thereto and/or heat may be applied to the tissue growth member 38 from a source surrounding the tissue growth member from, for example, a heater 106. Such heater 104 may apply convective heat and/or radiation type heat to the tissue growth member 38, as depicted by arrows 106. The heat applied to the tissue growth member 38 may, in part, attach the tissue growth member 38 to the occluder frame 36 (FIG. 5) by laminating at least some of the layers together. Further, the tissue growth member may, at least in part, be laminated by applying heat thereto for a predetermined period of time and at a predetermined heat, as known to one of skill in the art. Further, the lamination process applied to tissue growth member may be applied so that an external surface or the outer surface 110 of the tissue growth member 38 defines the texture 58, as previously set forth herein. As such, the second polymer sheet may include structural characteristics, such as a thickness, that facilitates the texture 58 along the outer surface 110 of the tissue growth member 38 upon the tissue growth member 38 being heated to form a laminated structure with the reinforcement textile 44 (FIG. 5) embedded therein. In another embodiment, the heat applied to the tissue growth member 38 may be primarily via convective and/or radiation type heat such that the lamination process may primarily be applicable to the second polymer sheet 42 being laminated over the reinforcement textile 44 of the tissue growth member 38. In either case, the outer surface 110 of the tissue growth member 38 may exhibit the texture 58, set forth herein, the texture 58 defined by the longitudinal and lateral structures 54, 56 of the reinforcement textile 44 (see FIG. 5) along an underside of the second polymer sheet 42, the texture 58 including textured longitudinal structures 55 and textured lateral structures 57. Such textured longitudinal and lateral structures 55, 57 may correspond with the longitudinal and lateral structures 54, 56, respectively, of the reinforcement textile 44 (FIG. 5). After the lamination process of the tissue growth member 38, the occluder frame 36 with the tissue growth member 38 attached thereto, may be removed from the mandril 96, after which, the implant 14 may be assembled by coupling the anchor frame 34 to the occluder frame 36, as depicted in FIG. 7. The anchor frame 34 of the anchor portion 32 may be coupled to the occluder frame 36 as previously described above, such that the anchor frame 34 may pivot between a deployed position (FIG. 1A) and a retracted position (FIG. 2A). In this manner, the implant 14 may be assembled as well as the tissue growth member 38 may be attached to the implant 14 with the before described texture 58 along an outer surface 110 of the tissue growth member 38.

With reference to FIGS. 5, 7 and 8, upon the lamination process being implemented, the before described recesses 60 may be formed in the outer surface of the tissue growth member 38, as shown in the enlarged view of FIG. 8. As previously described, the reinforcement textile 44 may include longitudinal structures 54 and lateral structures 56. Such longitudinal and lateral structures 54, 56 may extend in an overlapping manner relative to each other. For example, the longitudinal structures 54 may extend over and under adjacently extending lateral structures 56, as depicted. Similarly, the lateral structures 56 may extend over and under adjacently extending longitudinal structures 54. Upon the tissue growth member 38 undergoing the lamination process and receiving heat, the outer polymer sheet or second polymer sheet 42 may soften and form or compress against or slightly melt against the longitudinal and lateral structures 54, 56 of the reinforcement textile 44 to form the respective textured longitudinal and lateral structures 55, 57. This forming or melting process may create the recesses 60 defined in the outer surface 110 of the tissue growth member 38, or rather, the outer surface 110 of the second polymer sheet 42. Such recesses 60 may also be referenced as cavities, channels or openings that may also be defined as the before discussed texture 58 of the outer surface 110 of the tissue growth member 38. With this arrangement, the texture 58 and the recesses 60 defined in the outer surface 110 of the tissue growth member 38 may be sized and configured to assist in promoting endothelialization and, thus, sized and configured to promote tissue growth along the tissue growth member 38.

Further, each of the longitudinal structures 54 and the lateral structures 56 may define a spacing 112 between adjacently extending longitudinal structures 54 and adjacently extending lateral structures 56. In one embodiment, the spacing 112 between the respective longitudinal structures 54 and the respective lateral structures may be variable. In another embodiment, the spacing 112 between each of the longitudinal structure 54 and each of the lateral structures 56 may be generally consistent or generally constant. In another embodiment, the spacing 112 between adjacently extending longitudinal structures 54 may change over the length thereof because the longitudinal structures extend radially outward from the proximal side 48 to the occluder distal end 50. In another embodiment, the spacing 112 of the longitudinal and lateral structures 54, 56 may be larger adjacent a distal side of the occluder frame 36 so that the recesses 60 defined in the outer surface 110 adjacent the distal side of the implant 14 may be sized and configured to promote more cell growth and endothelialization for better long-term fixation of the implant 14 to tissue.

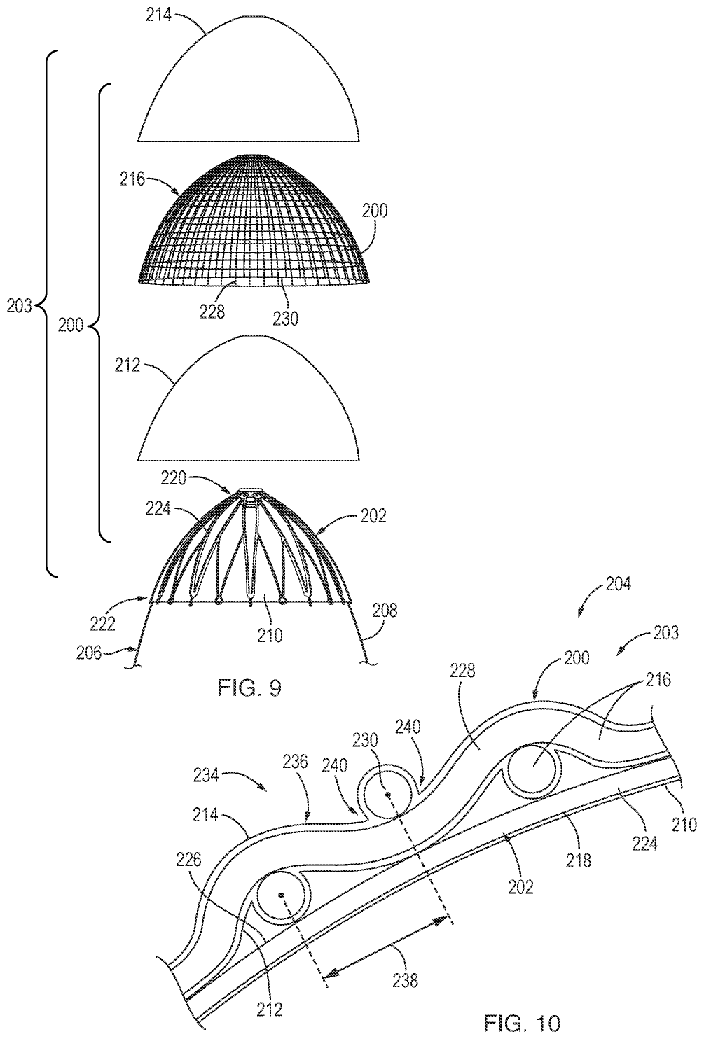

Now with reference to FIG. 9, another embodiment of a tissue growth member 200 coupled to a framework 202 to form an occluder portion 203 is provided. Similar to the previous embodiment described above, a mandril 206 may be sized and configured to allow for the tissue growth member 200 and framework 202 to be positioned thereon such that an occluder frame 224 is in an open position (or generally in the position that the occluder frame 224 is deployed) during formation and assembly. The mandril 206 may define a tip portion 208 that may be sized to fully support the occluder frame 224. The tissue growth member 200 may include multiple layers of varying thicknesses and materials configured to promote endothelialization. For example, in this embodiment, the tissue growth member 200 may include a first polymer sheet 210, a second polymer sheet 212, a third polymer sheet 214, and a reinforcement textile 216.

As previously set forth, the occluder portion 203 may be configured and assembled similarly to that described in the previous embodiment, such that the first polymer sheet 210 of the tissue growth member 200 may be positioned over the tip portion 208 of the mandril 206. The framework 202 of the implant 204 may be positioned to rest over the first polymer sheet 210 such that an inner side 218 of the framework 202 may extend along and against the first polymer sheet 210. Further, the framework 202 may extend radially over the first polymer sheet 210 from a proximal side 220 of the framework 202 to an occluder distal end 222 of the occluder frame 224.

With reference to FIGS. 9 and 10, in this embodiment, the tissue growth member 200 may include another polymer sheet, such as the second polymer sheet 212, positioned directly over the occluder frame 224. Further, in this embodiment, there may be multiple polymer sheets positioned over the occluder frame 224. For example, the second polymer sheet 212 and third polymer sheet 214 with the reinforcement textile 216 therebetween may each be positioned over the framework 202. The reinforcement textile 216 may be configured to reinforce and strengthen the tissue growth member 200. Further, similar to the previous embodiment, the reinforcement textile 216 may include longitudinal structures 228 and lateral structures 230 that define the reinforcement textile 216. Upon positioning the reinforcement textile 216 over the mandril 206 and over the occluder frame 224, the longitudinal structures 228 may extend from the proximal side 220 of the occluder frame 224 to adjacent the occluder distal end 222 of the occluder frame 224. The lateral structures 230 may extend laterally relative to the longitudinal structures 228 and around a circumference of the occluder frame 224. In this manner, the longitudinal structures 228 may extend in radially extending rows and the lateral structures 230 may extend laterally relative to the longitudinal structures 228, the lateral structures 230 each extending from adjacent ones of the lateral structures with a spacing 238 therebetween. As such, the longitudinal structures 228 and the lateral structures 230 may extend as an array or matrix pattern, similar to that described in the previous embodiment. With this arrangement, the reinforcement textile 216 may be sized and configured to reinforce and strengthen the first, second, and third polymer sheets 210, 212, 214 where the first, second, and third polymer sheets 210, 212, 214 may be relatively thin layers of the tissue growth member 200.

Further, similar to the previous embodiment, the occluder frame 224 and tissue growth member 200, being positioned on the tip portion 208 of the mandril 206, may be heated such that the tissue growth member 200 and the occluder frame 224 may be laminated to each other. The first polymer sheet 210 may be positioned directly adjacent to the inner side 218 of the framework 202 and the second polymer sheet 212 may be positioned adjacent to the outer side 226 of the occluder frame 224 and/or the reinforcement textile 216. Further, the second polymer sheet 212 and third polymer sheet 214 may each couple to the reinforcement textile 216, the second and third polymer sheets 212, 214 being formed to be on opposing sides of the reinforcement textile 216, thereby providing additional support and reinforcement to the tissue growth member 200. Similar to the previous embodiment, the outer polymer sheet or, in this case, the third polymer sheet 214 may define an outer surface 236 with a texture 234 (see texture 58 in FIGS. 6 and 7), upon the tissue growth member 200 being heated and undergoing the lamination process. Further, similar to the previous embodiment, the texture 234 of this embodiment may be defined by the longitudinal and lateral structures 228, 230 of the reinforcement textile 216 positioned under the third polymer sheet 214. As such, the outer surface 236 of the tissue growth member 200 of this embodiment may exhibit similar structural characteristics as the previous embodiment, as depicted in FIG. 7. In this manner, the tissue growth member 200 may include the additional polymer sheet, such as the second polymer sheet 212, which may provide additional support and reinforcement to the tissue growth member 200. Further, the texture 234 along the outer surface 236 of the tissue growth member 200 may exhibit similar structural characteristics as the texture 58 (FIGS. 6 and 7) described and depicted in the previous embodiment. For example, the texture 234 along the outer surface 236 of the tissue growth member 200 may define recesses 240 therein, similar to the previous embodiment, such that the recesses 240 may be created in the outer surface 236 upon laminating the outer polymer sheet, or third polymer sheet 214 to the reinforcement textile 216. Such recesses 240 defined in the outer surface 236 of the tissue growth member 200 may be sized and configured to promote endothelialization along the outer surface 236 of the tissue growth member, upon being implanted in, for example, the left atrial appendage of the heart.

In one embodiment, the first, second, and third polymer sheets 210, 212, 214 may be made of a polyurethane or PET material and the reinforcement textile 216 may be a fabric or polymeric material. Further, the first, second, and third polymer sheets 210, 212, 214 may be sized and configured to be relatively thin layers in comparison to the reinforcement textile 216 such that the tissue growth member 200 may be readily constricted with the implant 204 into the delivery system 12, as depicted in FIG. 3. In other embodiments, the first, second and third polymer sheets 210, 212, 214 may be different thicknesses to form the tissue growth member 200. In another embodiment, each of the first, second, and third polymer sheets 210, 212, 214 may vary in their size. In another embodiment, a first polymer sheet 210 may not be included such the second and third polymer sheets 212, 214 may be positioned over the outer side 226 of the occluder frame 224 with the reinforcement textile 216 extending therebetween.

The various structural components of the embodiments of the medical device system and the implant set forth herein may be formed from metallic materials, such as nitinol, stainless steel, titanium, or any other suitable metallic materials as well as any suitable polymeric materials, as known to one of skill in the art. Further, some components of the implant, such as the tissue growth member, may be formed from one or more polymeric materials or fabrics, such as polyurethane, PET, silicon or a copolymer thereof, or any other suitable biocompatible polymeric materials, as well as any other materials needed to form and manufacture the various components of the tissue growth member, as known by one of skill in the art. Further, the structural components of the various components of the medical device system and the implant may be formed by employing known manufacturing techniques and processes, such as lamination, gluing, stitching, bending, fastening, cutting, welding, soldering, etc., as known to one of skill in the art.

While the invention may be susceptible to various modifications and alternative forms, specific embodiments have been shown by way of example in the drawings and have been described in detail herein. Further, the structural features of any one embodiment disclosed herein may be combined or replaced by any one of the structural features of another embodiment set forth herein. However, it should be understood that the invention is not intended to be limited to the particular forms disclosed. Rather, the invention includes all modifications, equivalents, and alternatives falling within the spirit and scope of the invention as defined by the following appended claims.

What is claimed is:

1. A medical device system configured to occlude an opening in a heart, the medical device system comprising:
a delivery system including a handle and a catheter; and
an implant removably coupled to the catheter, the implant including a framework and a tissue growth member, the tissue growth member including:
an inner sheet positioned along an inner side of the framework and an outer sheet positioned along an outer side of the framework; and
a reinforcement textile positioned between the inner and outer sheets to define a texture along an outer surface of the tissue growth member.

2. The medical device system of claim 1, wherein the reinforcement textile extends with longitudinal structures and lateral structures, each one of the lateral structures extending transverse relative to each one of the longitudinal structures.

3. The medical device system of claim 1, wherein the reinforcement textile extends with longitudinal structures and lateral structures, each one of the longitudinal structures being spaced relative to adjacently extending ones of the longitudinal structures, each one of the lateral structures being spaced relative to adjacently extending ones of the lateral structures.

4. The medical device system of claim 1, wherein the reinforcement textile extends with longitudinal structures and lateral structures, the longitudinal structures positioned over the framework to extend radially outward from a proximal hub of the framework toward a distal end of the framework, the lateral structures positioned over the framework to extend transverse relative to the longitudinal structures.

5. The medical device system of claim 1, wherein the reinforcement textile extends with longitudinal structures and lateral structures, each one of the longitudinal structures extending over and under different ones of the lateral structures, each one of the lateral structures extending over and under different ones of the longitudinal structures.

6. The medical device system of claim 1, wherein the outer surface of the tissue growth member defines recesses therein such that structure that defines the recesses also defines the texture, the recesses being sized and configured to promote endothelialization along the outer surface of the tissue growth member.

7. The medical device system of claim 1, wherein the reinforcement textile and the outer sheet comprise a laminated structure, the laminated structure extending with the texture along the outer surface of the tissue growth member.

8. The medical device system of claim 1, wherein the tissue growth member comprises an intermediate sheet, the intermediate sheet positioned along the outer side of the framework and below the reinforcement textile.

9. A medical device configured to be percutaneously delivered with a delivery system to occlude an opening in a heart, the medical device comprising:
a framework extending radially outward from a proximal hub to a distal side of the framework; and
a tissue growth member attached to the framework, the tissue growth member including an outer polymeric sheet, and a reinforcement textile, the reinforcement textile including longitudinal structures and lateral structures, the longitudinal structures radially spaced from each other and each extending over the framework from the proximal hub toward the distal side of the framework, the lateral structures spaced from each other and positioned laterally relative to the longitudinal structures to each extend radially around a circumference of the radially extending framework, the outer polymeric sheet positioned over the reinforcement textile so that the longitudinal structures and the lateral structures define a texture along an outer surface of the tissue growth member.

10. The medical device of claim 9, wherein each one of the longitudinal structures extend over and under different ones of the lateral structures, and wherein each one of the lateral structures extend over and under different ones of the longitudinal structures.

11. The medical device of claim 9, wherein the outer surface of the tissue growth member defines recesses therein such that structure that defines the recesses also defines the texture, the recesses being sized and configured to promote endothelialization along the outer surface of the tissue growth member.

12. The medical device of claim 9, wherein the reinforcement textile and the outer polymeric sheet comprise a laminated structure, the laminated structure extending with the texture along the outer surface of the tissue growth member.

13. The medical device of claim 9, wherein the tissue growth member comprises an inner polymeric sheet, the inner polymeric sheet positioned along an inner side of the framework.

14. The medical device of claim 9, wherein the tissue growth member comprises an inner polymeric sheet and an intermediate polymeric sheet, the intermediate polymeric sheet and the outer polymeric sheet positioned along opposite sides of the reinforcement textile.

15. A method for occluding an opening in a heart, the method comprising:
advancing a medical device through a vasculature toward the opening in the heart with a pusher catheter such that the medical device is coupled adjacent a distal end of the pusher catheter;
positioning the medical device in the opening in the heart such that the medical device includes a framework and a tissue growth member, the tissue growth member including an outer polymeric sheet and a reinforcement textile, the reinforcement textile including longitudinal structures and lateral structures, the longitudinal structures radially spaced from each other and each extending over the framework from the proximal hub toward the distal side of the framework, the lateral structures spaced from each other and positioned laterally relative to the longitudinal structures to each extend radially around a circumference of the radially extending framework, the outer polymeric sheet positioned over the reinforcement textile so that the longitudinal structures and the lateral structures define a texture along an outer surface of the tissue growth member; and
anchoring the medical device within the opening of the heart with tines extending from the framework so that the texture of the tissue growth member is positioned against tissue defining the opening in the heart.

16. The method according to claim 15, wherein the positioning comprises positioning the medical device such that the tissue growth member includes each one of the longitudinal structures to extend over and under different ones of the lateral structures, and each one of the lateral structures to extend over and under different ones of the longitudinal structures.

17. The method according to claim 15, wherein the anchoring comprises positioning recesses defined in the outer surface of the tissue growth member against tissue in the heart.

18. The method according to claim 15, wherein the positioning comprises positioning the medical device with a laminated structure extending with the texture along the outer surface of the tissue growth member, the laminated structure extending with at least the reinforcement textile and the outer polymeric sheet.
